(19) United States
(12) Patent Application Publication
(10) Pub. No.: US 2016/0068655 A1
(43) Pub. Date: Mar. 10, 2016

(54) INTEGRATED PROCESS FOR THE PRODUCTION OF BENZOATE PLASTICIZERS

(71) Applicant: Eastman Chemical Company, Kingsport, TN (US)

(72) Inventors: Pinguan Zheng, Johnson City, TN (US); Jonathan J. Mabe, Chestertown, MD (US); Charles Edwan Sumner, JR., Kingsport, TN (US); Mesfin Ejerssa Janka, Kingsport, TN (US); Beverly Lenora Frye, Kingsport, TN (US); Stephanie Rollins Testerman, Kingsport, TN (US); Brent Alan Tennant, Kingsport, TN (US)

(73) Assignee: EASTMAN CHEMICAL COMPANY, Kingsport, TN (US)

(21) Appl. No.: 14/940,237

(22) Filed: Nov. 13, 2015

(51) Int. Cl. C08K 5/103; C07C 67/60; B01J 31/22; C07C 67/08; C08K 5/105; B01J 27/232; C07C 67/00; C07C 51/265

(52) U.S. Cl. CPC C08K 5/103; C07C 67/00; C07C 67/60; C07C 51/265; C07C 67/08; C08K 5/105; B01J 27/232; B01J 31/2208

(57) ABSTRACT

The invention relates to a process that integrates the oxidation of toluene to benzoic acid with the production of benzoate plasticizers. Toluene is fed to an oxidation vessel in the presence of oxygen and an oxidation catalyst wherein benzoic acid serves as the solvent for the oxidation. The crude benzoic acid produced is not purified and is then reacted with an alcohol in the presence of an esterification catalyst to produce the crude benzoate ester. The oxidation catalyst, esterification catalyst, and other impurities can be mostly removed from the crude benzoate ester in subsequent washing and filtering steps. The benzoate esters produced through this method can be made in fewer steps with both yields and purities above 80%.

### FIELD OF THE INVENTION

The invention generally relates to the synthesis of benzoate esters, in particular, to a process that integrates the oxidation of toluene to benzoic acid with the esterification of benzoic acid to produce benzoate plasticizers.

### BACKGROUND OF THE INVENTION

Benzoic acid is an important raw material with an annual world consumption of nearly 500 thousand metric tons and is forecasted to grow at an average annual rate of about 3% per year until at least 2018. Benzoic acid is widely used in the production of food preservatives and benzoate plasticizers. This world consumption of benzoic acid relies on the sodium and potassium salts of benzoate used in food and beverage preservation but a considerable amount is also used for benzoate plasticizers. The benzoate plasticizer market continued to increase at an average annual rate of 10.7% between 2009 and 2013, this growth is largely driven by the replacement of phthalate plasticizers in numerous applications due to regulatory control and their potential toxicity.

The manufacture of benzoic acid involves the liquid phase oxidation of toluene in the presence of a cobalt catalyst with or without a manganese catalyst. Typical byproducts include benzaldehyde, benzyl alcohol, benzyl benzoate, and biphenyls. In the current commercial manufacturing process, unrefined benzoic acid is generally purified by multiple distillations to remove oxidation byproducts and residual traces of catalyst. Research has been presented that uses an oxidative approach to selectively remove organic byproducts using chlorine under caustic conditions wherein the benzoate alkali salt was converted back to benzoic acid with the treatment of a strong mineral acid. One other approach used in the art to purify benzoic acid is the esterification of benzoic acid to a methyl benzoate wherein the methyl benzoate was purified through fractional distillation and then converted back to the benzoic acid through saponification with a strong acid. In many cases, extractions and crystallization are employed to deliver good quality benzoic acid.

Currently, benzoate plasticizers are manufactured using purified benzoic acid as a feed. The purification of benzoic acid involves a number of distillation steps which make the current benzoate plasticizers production very expensive.

There is a need in the chemical industry for an inexpensive and high yield process to make benzoate plasticizers. Such a process would be able to directly convert toluene to a benzoate ester product through subsequent oxidation and esterification reactions without the need to purify between steps. This process would be able to significantly reduce the cost of benzoate plasticizers and simplify both the process and feasibility of an integrated benzoic acid/esterification process.

Herein is described a process of converting toluene to useful benzoate esters by eliminating tedious and costly multiple-step distillations in the refining of benzoic acid, thus simplifying the benzoic acid process and reducing manufacturing costs. This invention enables the integration of the toluene oxidation to benzoic acid process with catalytic esterification to directly convert unrefined benzoic acid into desired benzoate esters with both good yields and quality through the effective removal of residual catalysts and light impurities.

### SUMMARY OF THE INVENTION

The present invention provides in a first embodiment a method for producing benzoate esters comprising: contacting toluene in an oxidation vessel with a benzoic acid solvent, an oxygen containing gas, and an oxidation catalyst to produce a crude benzoic acid product; transferring the crude benzoic acid product to an esterification vessel where the crude benzoic acid product is contacted with an alcohol, an esterification catalyst, and an azeotropic agent to produce a crude benzoate ester product; and treating the crude benzoate ester product to produce a benzoate ester with a reduced impurity profile.

The present invention provides in a second embodiment a method for producing benzoate esters comprising: contacting toluene in an oxidation vessel with a benzoic acid solvent, an oxygen containing gas, and an oxidation catalyst to produce a crude benzoic acid product; flashing the crude benzoic acid product to give a purified benzoic acid; contacting the purified benzoic acid in an esterification vessel with an alcohol, an esterification catalyst, and an azeotropic agent to produce a crude benzoate ester product; treating the crude benzoate ester product to produce a benzoate ester with a reduced impurity profile.

The present invention provides in a third embodiment a method for producing benzoate ester plasticizers comprising: contacting toluene in an oxidation vessel with a benzoic acid solvent, an oxygen containing gas, and an oxidation catalyst to produce a crude benzoic acid product; feeding the crude benzoic acid product to an esterification vessel where the crude benzoic acid product is contacted with an alcohol, an esterification catalyst, and an azeotropic agent to produce a crude benzoate ester plasticizer; washing the crude benzoate ester plasticizer with a caustic treatment comprising sodium carbonate and sodium hydroxide; and filtering the crude benzoate ester plasticizer with a filtration aid to produce a benzoate ester plasticizer with a reduced impurity profile.

### BRIEF DESCRIPTION OF THE DRAWINGS

FIG. 1 is a flow chart showing an exemplary batch fed reactor set up for the oxidation of toluene.

FIG. 2 is a flow chart showing an exemplary continuous feed reactor set up for the oxidation of toluene.

FIG. 3 is drawing showing a representative lab setup for the esterification of crude benzoic acid to make a benzoate ester.

FIG. 4 is a flow chart showing the process for the manufacture of benzoate esters from unrefined benzoic acid.

FIG. 5 is a flow chart showing the integrated oxidation/esterification process to produce benzoic esters.

### DETAILED DESCRIPTION OF THE INVENTION

It has been surprisingly discovered that benzoate esters can be efficiently prepared from the oxidation of toluene followed by a direct esterification of the crude benzoic acid without the need for distillations or additional purification steps for the respective reagents. Employing this method simplifies the benzoate ester process by reducing manufacturing costs by directly converting unrefined benzoic acid from the oxidation of toluene into benzoate esters in high yields through the effective removal of residual catalysts and light impurities.

The present invention provides in a first embodiment a method for producing benzoate esters comprising: contacting toluene in an oxidation vessel with a benzoic acid solvent, an oxygen containing gas, and an oxidation catalyst to produce a crude benzoic acid product; transferring the crude benzoic acid product to an esterification vessel where the crude benzoic acid product is contacted with an alcohol, an esterification catalyst, and an azeotropic agent to produce a crude benzoate ester product; and treating the crude benzoate ester product to produce a benzoate ester with a reduced impurity profile.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, each numerical parameter should at least be construed in light of the number of reported significant digits and by applying ordinary rounding techniques. Further, the ranges stated in this disclosure and the claims are intended to include the entire range specifically and not just the endpoint(s). For example, a range stated to be 0 to 10 is intended to disclose all whole numbers between 0 and 10 such as, for example 1, 2, 3, 4, etc., all fractional numbers between 0 and 10, for example 1.5, 2.3, 4.57, 6.1113, etc., and the endpoints 0 and 10. Also, a range associated with chemical substituent groups such as, for example, “C₁ to C₅ hydrocarbons”, is intended to specifically include and disclose C₁ and C₅ hydrocarbons as well as C₂, C₃, and C₄ hydrocarbons.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

It is to be understood that the mention of one or more process steps does not preclude the presence of additional process steps before or after the combined recited steps or intervening process steps between those steps expressly identified. Moreover, the lettering of process steps or ingredients is a convenient means for identifying discrete activities or ingredients and the recited lettering can be arranged in any sequence, unless otherwise indicated.

As used herein the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

The first embodiment of the current invention comprises contacting toluene in an oxidation vessel with a benzoic acid solvent, an oxygen containing gas, and an oxidation catalyst to produce an unpurified crude benzoic acid product.

The oxidation process can be conducted under continuous, semi-continuous, and batch modes of operation and may utilize a variety of oxidation vessels or reactor types. Examples of suitable oxidation vessels or reactor types include, but are not limited to, stirred tank, continuous stirred tank, trickle bed, tower, slurry, and tubular reactors.

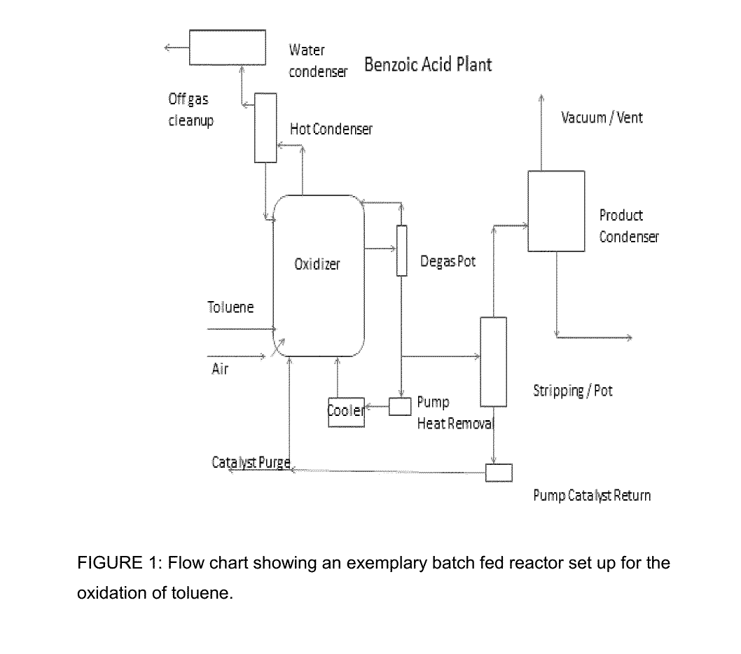

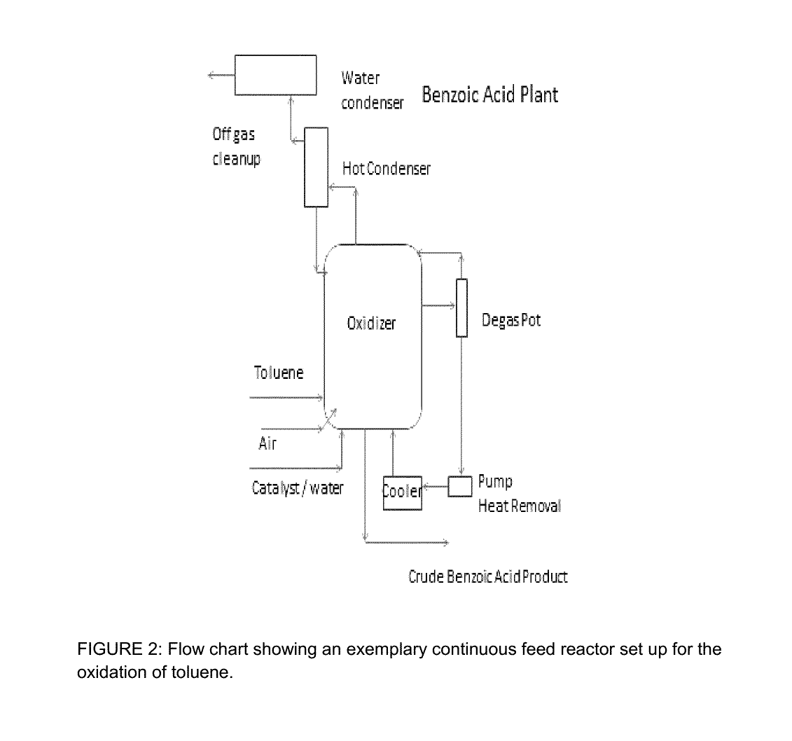

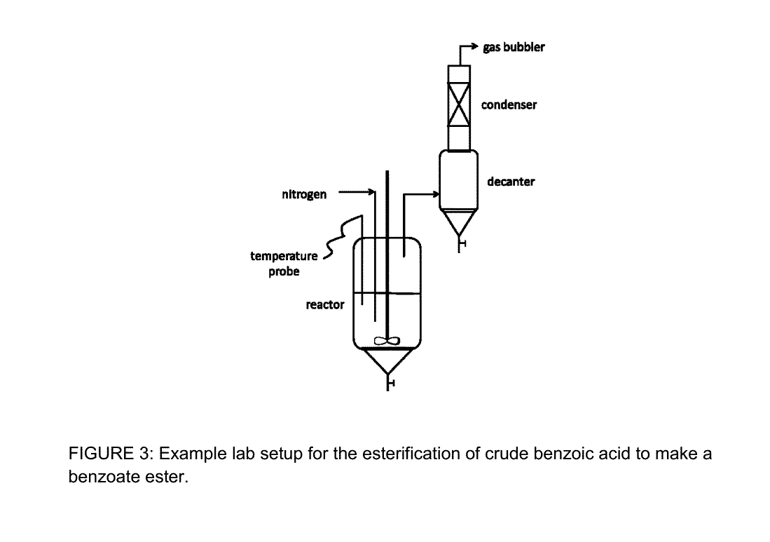

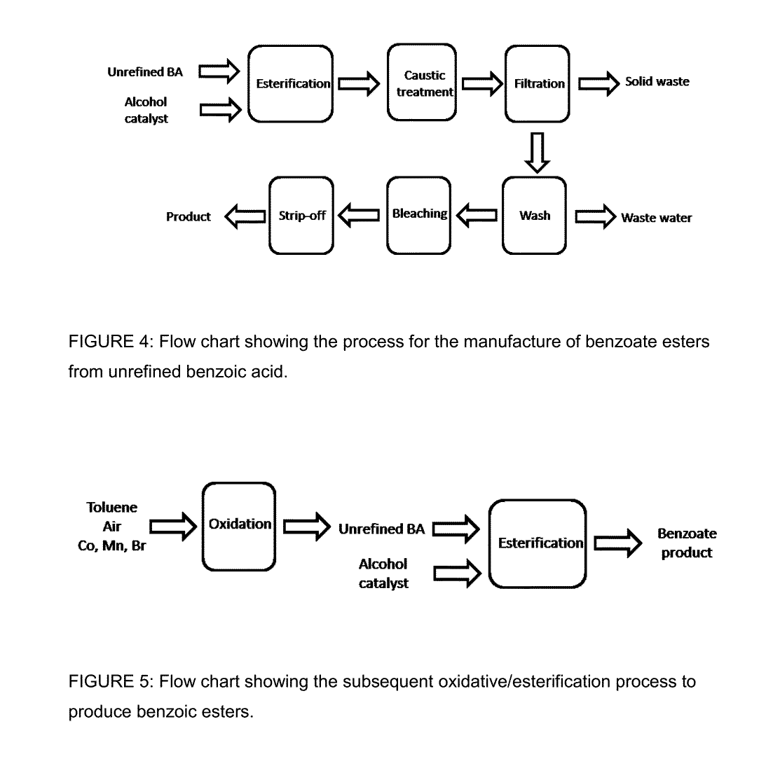

In some examples, the oxidation reaction can be run as a fed batch reaction (FIG. 1) where toluene, diluted oxygen, and an oxidation catalyst are fed to a stirred tank containing a sufficient amount of benzoic acid to maintain a homogeneous mixture. Alternatively, in other embodiments, the toluene and diluted oxygen are fed continuously to the agitated vessel containing benzoic acid and oxidation catalyst (FIG. 2). The water produced as a byproduct of the reaction is removed as a vapor with the oxidation off gas through a hot condenser.

The oxygen containing gas can be fed to the oxidation reactor before, during, and/or after the other reagents and/or components are added. Examples of oxygen containing gases include, but are not limited to, air, an inert carrier gas such as nitrogen with added oxygen, or gases recycled from the process. The concentration of oxygen in the oxygen containing gas can be in the range from 0.5 to 30 mole %, from 0.5 to 25 mole %, from 0.5 to 20 mole %, from 0.5 to 15 mole %, from 0.5 to 10 mole %, or from 3 to 4 mole %.

The oxidation catalyst comprises a cobalt salt, a manganese salt, and a bromine compound that can liberate ionic bromide or HBr under reaction conditions. The cobalt salt can include, but is not limited to, cobalt acetate, cobalt hydroxide, cobalt carbonate, cobalt benzoate, and combinations thereof. The manganese salt can include, but is not limited to, manganese acetate, manganese carbonate, manganese benzoate, and combinations thereof. The cobalt and/or manganese salts used as the oxidation catalyst produce a soluble form of the catalyst in the reaction mixture. The combined concentrations of cobalt and manganese useful for the oxidation reaction begin at about 50 ppm and can increase in concentration. Other examples of the combined cobalt and manganese concentrations range from 50-500 ppm, 50-400 ppm, 50-300 ppm, 50-200 ppm, and 50-100 ppm.

The ratio of Co to Mn used as the oxidation catalyst can be determined based on the temperature of the oxidation reaction. When a higher temperature range is used, for example 170-190° C., a Co/Mn ratio of 0.1-1.0 produces better oxidation yields. When a lower temperature range is used, for example 140-170° C., a Co/Mn ratio of 1.0-0.05 produces better oxidation yields. Due to its corrosive nature, the concentration of bromine is kept low to maintain a smooth oxidative conversion of toluene. While the oxidation reaction will tolerate very high bromine concentrations, the bromine concentration is normally kept in the range from 75-250 ppm.

The unpurified crude benzoic acid produced from the oxidation of toluene may have a variety of impurities. Impurities can include, but are not limited to, benzyl benzoate, benzyl alcohol, benzaldehyde, biphenyl, biphenyl-2-carboxylic acid, biphenyl-3-carboxylic acid, biphenyl-4-carboxylic acid, fluorenone, phenoxymethyl benzoate, and 3,4-benzocoumarin. The total biphenyls is the sum of the weight percentage of biphenyl, biphenyl-2-carboxylic acid, biphenyl-3-carboxylic acid, and biphenyl-4-carboxylic acid. The amount of impurities in the unpurified crude benzoic acid can vary with the percent conversion of the oxidation of toluene to benzoic acid.

Byproducts produced from the toluene oxidation reaction are best minimized by controlling the temperature and catalyst compositions used in the reaction. Biphenyl compounds are one group of impurities that can be at least partially controlled with temperature and choice of catalyst. The biphenyl compounds and other impurities produced during the oxidation step can be attenuated by adjusting the concentration of the cobalt catalyst and the amount of oxygen gas exiting the reactor. For example, benzocoumarin and carboxybiphenyls are produced in the greatest amount when the cobalt concentration is about 300 ppm. In order to maintain high conversions at low cobalt concentrations of 50-100 ppm, the concentration of manganese needs be increased. With higher manganese concentrations the temperature of the oxidation reaction can be increased to 180° C. or higher. Conversely, if the cobalt concentration is left high, the reaction temperature should be kept from 140-160° C. to minimize formation of these biphenyl byproducts. For example, the rate at which biphenyl derivatives are produced during the oxidation is proportional to the reaction temperature for a constant cobalt concentration.

It is important that both a high conversion and selectivity is obtained for the oxidation of toluene to benzoic acid in order to avoid the production of volatile byproducts such as benzyl benzoate, benzyl alcohol, and benzaldehyde, in addition to the biphenyl compounds mentioned above. The presence of such compounds in the product of the oxidation can lead to odor and other quality problems with the benzoate ester. Thus, the conversion of toluene to benzoic is typically greater than 80%, 90%, 95%, 98%, or 99%. In addition to a high conversion, a high selectivity to benzoic acid is also desired so fewer impurities exist that can be carried over to the esterification reaction. The selectivity or purity for the oxidation of toluene to benzoic acid is typically greater than 80%, 90%, 95%, 98%, or 99%.

The crude benzoic acid produced from the oxidation of toluene may have a variety of different compositions. For example, in some embodiments, the oxidation of toluene to crude benzoic acid can be performed with at least a 99% conversion and with less than 0.3 weight %, less than a 0.2 weight %, or less than a 0.1 weight % total biphenyls and less than a 0.2 weight %, or less than a 0.1 weight %, or less than a 0.05 weight % benzaldehyde, fluoenone, benzyl benzoate, and phenoxymethyl benzoate. Total biphenyls is the sum of the weight percentage of biphenyl, biphenyl-2-carboxylic acid, biphenyl-3-carboxylic acid, and biphenyl-4-carboxylic acid. Weight percentage, as used here, is the weight percentage of the total weight of all oxidation products resulting from the oxidation of toluene including any unreacted toluene.

When the desired amount of toluene is converted to benzoic acid in the oxidation vessel, a portion of the unpurified crude benzoic acid product is transferred to an esterification vessel wherein the unpurified crude benzoic acid is then reacted with an alcohol to produce an unpurified crude benzoic ester product and/or a unpurified crude dibenzoate ester product. The portion of unpurified crude benzoic acid product transferred to the esterification vessel may be greater than 25 weight %, 50 weight %, 75 weight %, 90 weight %, 95 weight %, or 99 weight % of the total product mixture. The remaining unpurified crude benzoic acid product that is not transferred to the esterification vessel may be recycled and reintroduced into the oxidation reactor to be used as a solvent for subsequent oxidations of toluene. This recycled crude benzoic ester product may have less than 0.5 weight %, less than a 0.4 weight %, or less than a 0.3 weight % total biphenyls and less than a 0.2 weight %, less than a 0.1 weight %, less than a 0.075 weight %, or less than a 0.05 weight % benzaldehyde, fluoenone, benzyl benzoate, and phenoxymethyl benzoate. Total biphenyls is the sum of the weight percentage of biphenyl, biphenyl-2-carboxylic acid, biphenyl-3-carboxylic acid, and biphenyl-4-carboxylic acid. Weight percentage, as used here, is the weight percentage of the total weight of all the oxidation products resulting from the oxidation of toluene including any unreacted toluene.

The first embodiment of the present invention includes the step of feeding the unpurified crude benzoic acid to an esterification vessel where the unpurified crude benzoic acid product is contacted with an alcohol, an esterification catalyst, and an azeotropic agent to produce a crude benzoate ester product.

The esterification process can be conducted under continuous, semi-continuous, and batch modes of operation and may utilize a variety of esterification vessels or reactor types. Examples of suitable esterification vessels or reactor types include, but are not limited to, stirred tank, continuous stirred tank, trickle bed, tower, and reactive distillation columns.

In some examples, the esterification reaction can be run as a batch reaction where the crude benzoic acid product, the alcohol, the esterification catalyst, and the azeotropic agent are fed to a stirred tank to maintain a homogeneous mixture. Alternatively, in other embodiments, the crude benzoic acid product and the alcohol are fed continuously to an agitated vessel containing the azeotropic agent and esterification catalyst. The water produced as a byproduct of the esterification reaction is removed as a vapor.

The esterification catalyst comprises tin compounds, titanium compounds, zinc compounds, zirconium compounds, or combinations thereof. Esterification employs 0.01 to 5 weight % relative to the amount of benzoic acid of at least one or two metal catalysts comprising tin, titanium, zinc and zirconium. Some examples of the esterification catalyst include, but are not limited to, titanium (IV) isopropoxide, stannous oxalate, or combinations thereof. The weight percentage of titanium (IV) isopropoxide used for the esterification reaction can range from 0.1 to 2.5 weight %, from 0.01 to 1 weight %, from 0.01 to 0.50 weight %, and from 0.01 to 0.10 weight % relative to the weight of benzoic acid. The weight percentage of stannous oxalate used for the esterification reaction can range from 0.1 to 1.0 weight %, from 0.01 to 0.25 weight %, from 0.01 to 0.10 weight %, and from 0.01 to 0.05 weight % relative to the weight of benzoic acid.

The alcohol comprises at least one of the following structures:

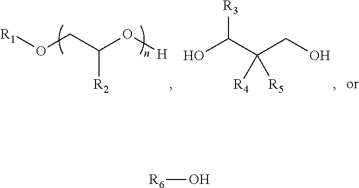

wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, R₅ is a hydrogen, methyl, or ethyl group, and R₆ is an alkyl group having 1 to 18 carbon atoms. In other embodiments, R₁ is an alkyl group having 3 to 8 carbons, an alkyl group having 4 to 8 carbons, an alkyl group having 5 to 8 carbons, or an alkyl group having 6 to 8 carbons. In other embodiments, R₃ is an alkyl group having 2 to 5 carbons, an alkyl group having 3 to 5 carbons, or an alkyl group having 4 to 5 carbons. In other embodiments, R₄ is an alkyl group having 2 to 4 carbons, or an alkyl group having 3 to 5 carbons. In other embodiments, R₆ is an alkyl group having 6 to 18 carbons, an alkyl group having 9 to 18 carbons, an alkyl group having 12 to 18 carbons, or an alkyl group having 15 to 18 carbons. In some examples, the alcohol comprises stearyl alcohol, diethylene glycol, dipropylene glycol, ethylene glycol 2-ethylhexyl ether, diethylene glycol 2-ethylhexyl ether, diethylene glycol monomethyl ether, 1,4-cyclohexanedimethanol (CHDM), 2,2,4-trimethypentage-1,3-diol, or a combination thereof.

The term “benzoate ester”, as used herein, broadly refers to benzoate esters, dibenzoate esters, benzoate plasticizers, and dibenzoate plasticizers. These benzoate esters, dibenzoate esters, benzoate plasticizers, and dibenzoate plasticizers categories of molecules are all encompassed by the term “benzoate ester” used in this application unless specifically referred to separately.

Depending on the concentration of reagents and the functionality (f=1, 2, . . . ) of the alcohol used for the esterification reaction, a number of different benzoate esters and dibenzoate esters can be synthesized. The following structures represent the general form of the benzoate and dibenzoate compounds that can be produced.

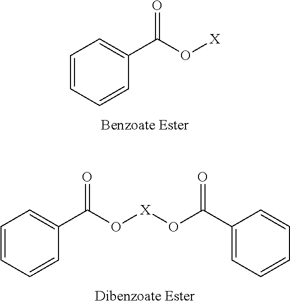

The X group corresponds to any chemical group or radical that would represent the respective moiety for any of the previously mentioned alcohols. For example, if the alcohol used in the esterification reaction was ethyl alcohol (f=1), X would be an ethyl group or —CH₂CH₃ group in the benzoate ester. If the alcohol used in the esterification reaction was ethylene glycol (f=2), X would be an ethyl group, or —CH₂CH₂—, in the dibenzoate ester product or an ethoxy group, or —CH₂CH₂OH, in the benzoate ester depending on the stoichiometry or reaction conditions used in the esterification reaction.

The term “azeotropic agent”, as used herein, refers to a liquid added to form a mixture of two or more substances which behaves like a single substance in that the vapor produced by partial evaporation of the liquid has the same composition as the liquid. The constant boiling mixture exhibits either a maximum or minimum boiling point as compared with the other mixtures of the same substances. The azeotropic agent added during the esterification typically makes up 0 to 25 weight % of the reaction mass and comprises petroleum distillate, toluene, isopar C, xylene, isooctane, or a combination thereof. In some embodiments the azeotropic agent is isopar C or toluene.

It is important that both a high conversion and product purity is obtained for the esterification of crude benzoic acid to benzoate esters or dibenzoate esters. Thus, the esterification of benzoic acid typically has a conversion greater than 80%, 90%, 95%, 98%, or 99%. In addition to a high conversion, a high purity of the desired benzoic ester is also desired. The purity of esterification of crude benzoic acid is typically greater than 80%, 90%, 95%, 98%, or 99%.

The final diglycol dibenzoate ester product may have less than a 0.3 area %, less than a 0.07 area %, less than a 1.3 area %, less than a 0.1 area %, less than a 2.2 area %, less than a 0.7 area %, or less than a 0.2 area % benzaldehyde, benzyl alcohol, total biphenyls, benzophenone, benzyl benzoate, benzo coumarin, and brominated benzoate esters. Other benzoate esters may have less than a 19.0 area %, less than 2.0 area %, or less than a 2.2 area % of unreacted alcohol, total biphenyls and benzyl benzoate. Area percentage, as used herein, is the area percentage of the total area of all GC peaks resulting from the esterification including unreacted alcohol.

The esterification reaction is performed at a pressure from 100 to 900 mmHg or from 100 to 500 mmHg combined with a temperature from 50 to 250° C., a temperature from 150 to 250° C., or a temperature from 170 to 220° C. One key to high conversion of the esterification reaction is to continuously remove water from the reaction system, thereby driving the equilibrium to product. Techniques include, but are not limited to, the use of an azeotropic agent, nitrogen purge (0.05 to 1.0 scfh) at a pressure of 760 to 900 mmHg, and vacuum (100 torr to 500 mmHg).

The first embodiment of the present invention includes the step of treating the crude benzoate ester product to produce a benzoate ester with a reduced impurity profile.

The term “treating”, as used herein, refers to the step of working up the crude benzoate ester product, and comprises at least one of the following: the caustic treatment of the crude benzoate ester product; a filtration step; a wash step; a decolorization step; and/or a strip-off step.

The term “reduced impurity profile”, as used herein, refers to a benzoate ester product that has a higher total percentage weight of the final benzoate ester product composition relative to the crude benzoate ester product composition. This “reduced impurity profile” is the result from the caustic treatment of the crude benzoate ester product; the filtration of the crude benzoate ester product; the washing the crude benzoate ester product; the decolorization of the crude benzoate ester product, the strip-off of the crude benzoate ester product, or any combination thereof.

The caustic treatment of the crude benzoate ester product is carried out at a temperature from 50 to 100° C. using a base at a concentration of 1 to 30 weight % in water. Examples of suitable bases include, but are not limited to, sodium carbonate, sodium hydroxide, sodium bicarbonate, potassium carbonate, potassium hydroxide, and potassium bicarbonate. In some embodiments, the base is a 26 weight % aqueous sodium carbonate solution or a 10 weight % sodium hydroxide aqueous solution. The crude benzoate ester is washed with water, preferably at a pH greater than 7, to remove the water soluble impurities including the residual oxidation catalyst and/or the residual esterification catalyst.

The filtration and wash step is performed by running the crude benzoate ester product through a pad of a porous filtration aid. Examples of porous filtration aides include, but are not limited to: celite diatomite comprising silicon dioxide (80 to 90 weight %), aluminum oxide (2 to 4 weight %), and iron oxide (0.5 to 2 weight %); perlite comprising silicon dioxide (70 to 75 weight %), aluminum oxide (12 to 15 weight %); cellulose; and activated carbon.

The decolorization or bleaching step is performed at a temperature from 50 to 100° C. using a bleaching agent and/or activated carbon. Typical decolorization or bleaching agents can be used such as peroxide-based bleaches (i.e. hydrogen peroxide, sodium percarbonate, sodium perborate) and chlorine-based bleaches (i.e. sodium hypochlorite and calcium hypochlorite). In some embodiments, the decolorization or bleaching agent is hydrogen peroxide.

The strip-off step is used to remove volatile impurities. Examples of practicing the strip-off step may include a steam strip-off or a vacuum strip-off at 10 to 50 mmHg.

Benzoate esters can be used as stable plasticizers primarily to increase the plasticity or fluidity of a material. These benzoate esters have an excellent degree of compatibility in numerous polymer systems which allow their use at various concentrations to achieve a variety of desired properties. This family of plasticizers demonstrates excellent inert filler acceptance that provides wear characteristics and lower formulation costs for cast polymers. Benzoate esters can contribute to improved tear strength, better rebound, and reduced swell with certain solvents and are adaptable to both metering and hand batch polymer mix systems. Common uses for these types of benzoate ester or dibenzoate ester plasticizers include, but are not limited to, business machine rolls, printing rolls, duplicating rolls, as well as gaskets and seals. Any of the benzoate ester and/or dibenzoate ester products made with this invention may be incorporated into a polymer material.

The second embodiment of the current invention is a method for producing benzoate esters comprising: contacting toluene in an oxidation vessel with a benzoic acid solvent, an oxygen containing gas, and an oxidation catalyst to produce a crude benzoic acid product; flashing the crude benzoic acid product to give a purified benzoic acid; contacting the purified benzoic acid in an esterification vessel with an alcohol, an esterification catalyst, and an azeotropic agent to produce a crude benzoate ester product; treating the crude benzoate ester product to produce a benzoate ester with a reduced impurity profile.

It is understood that the descriptions outlining and teaching the method of producing a benzoate ester previously discussed, which can be used in any combination, apply equally well to the second embodiment of the invention teaching a method to produce benzoate esters.

In the first embodiment, when the desired amount of toluene is converted to benzoic acid in the oxidation vessel, a portion of the unpurified crude benzoic acid product is transferred to an esterification vessel wherein the unpurified crude benzoic acid is then reacted with an alcohol to produce an unpurified crude benzoic ester product and/or a unpurified crude dibenzoate esters product. As an alternative in this second embodiment, a stream of the hot reaction mixture from the oxidation vessel is removed from the oxidation reactor and a portion of that stream is fed to a stripping pot where a majority of the crude benzoic acid product is flashed to a receiving tank to give a purified benzoic acid that is subsequently fed to the esterification vessel. This flash step, although an additional step, allows for a simple, quick, and efficient way to partially purify the benzoic acid reagent. The remaining crude benzoic acid portion in the stripping pot can then be cooled and returned to the oxidation vessel. In some embodiments, the toluene feed can optionally be diluted with this returned portion of the crude benzoic acid stream from the stripping pot. The residue or crude benzoic acid from the stripping pot can additionally be treated to recover the oxidation catalyst for reuse and to purge byproducts.

In some examples, the majority of the crude benzoic acid product from the oxidation reaction can be distilled or flashed from the oxidation vessel with no fractionation into a receiving tank. This purified benzoic acid product is then transferred from the receiving tank to the esterification vessel or esterification reactor where it is converted to the crude benzoate ester product.

In other examples, the toluene and diluted oxygen are fed continuously to the agitated oxidation vessel containing benzoic acid and oxidation catalyst (FIG. 2). The water byproduct produced from the oxidation reaction is removed as a vapor with the oxidation off gas through a hot condenser.

The crude benzoic acid product can be flashed and transferred to the esterification vessel. Using such a flash method provides a means for removing some of the undesired products and/or impurities in the crude benzoic acid product. Flashing the crude benzoic acid product comprises taking a stream of the hot reaction mixture containing the crude benzoic acid, removing it from the oxidation reactor, and feeding a portion of that crude benzoic acid stream to a stripping pot where the crude benzoic acid product is flashed to a receiving tank to form a purified benzoic acid product. The term, “flash”, as used herein, broadly refers to a single-stage batch or continuous operation where a liquid mixture is partially vaporized under atmosphere pressure or reduced pressure wherein the vapor produced and the residual liquid are in equilibrium. The flashed vapor goes through the condenser and is removed from the system. The purified benzoic acid product is then subsequently fed to the esterification reactor. The remaining portion is cooled and returned to the oxidation reactor. The toluene feed can optionally be diluted with this returned portion of the stream. The residue from the stripping pot is treated to recover the oxidation catalyst for reuse and to purge byproducts.

The purified benzoic acid product produced from the oxidation of toluene may have a variety of different compositions. For example, in some embodiments, the purified benzoic acid product will have less than 0.3 weight %, less than a 0.2 weight %, or less than a 0.1 weight % total biphenyls and less than a 0.2 weight %, or less than a 0.1 weight %, or less than a 0.05 weight % benzaldehyde, fluoenone, benzyl benzoate, and phenoxymethyl benzoate. Total biphenyls is the sum of the weight percentage of biphenyl, biphenyl-2-carboxylic acid, biphenyl-3-carboxylic acid, and biphenyl-4-carboxylic acid. Weight percentage, as used here, is the weight percentage of the total weight of all oxidation products resulting from the oxidation of toluene including any unreacted toluene.

When the desired amount of toluene is converted to benzoic acid in the oxidation vessel, a portion of the unpurified crude benzoic acid product may be flashed to a receiving tank and then transferred to an esterification vessel wherein the purified crude benzoic acid is then reacted with an alcohol to produce an unpurified crude benzoic ester product and/or a unpurified crude dibenzoate ester product. The portion of unpurified crude benzoic acid product flashed to the receiving tank may be greater than 25 weight %, 50 weight %, 75 weight %, 90 weight %, 95 weight %, or 99 weight % of the total product mixture. The remaining unpurified crude benzoic acid product that is not transferred to the esterification vessel may be recycled and reintroduced into the oxidation reactor to be used as a solvent for subsequent oxidations of toluene. The purified benzoic acid product that was flashed from the crude benzoic acid product may be recycled and reintroduced into the oxidation reactor to be used as a solvent for subsequent oxidations of toluene. In some examples, both a portion of the crude benzoic acid product and the purified benzoic acid product may be recycled and reintroduced into the oxidation reactor to be used as a solvent for subsequent oxidations of toluene. Both the recycled crude benzoic acid product and the purified benzoic acid product may have less than 0.5 weight %, less than a 0.4 weight %, or less than a 0.3 weight % total biphenyls and less than a 0.2 weight %, less than a 0.1 weight %, less than a 0.075 weight %, or less than a 0.05 weight % benzaldehyde, fluoenone, benzyl benzoate, and phenoxymethyl benzoate. Total biphenyls is the sum of the weight percentage of biphenyl, biphenyl-2-carboxylic acid, biphenyl-3-carboxylic acid, and biphenyl-4-carboxylic acid. Weight percentage, as used herein, is the weight percentage of the total weight of all the oxidation products resulting from the oxidation of toluene including any unreacted toluene.

The esterification process can be conducted under continuous, semi-continuous, and batch modes of operation and may utilize a variety of esterification vessels or reactor types. Examples of suitable esterification vessels or reactor types include, but are not limited to, stirred tank, continuous stirred tank, trickle bed, tower, slurry, and tubular reactors.

In some examples, the esterification reaction can be run as a batch reaction where the purified benzoic acid product, the alcohol, the esterification catalyst, and the azeotropic agent are fed to a stirred tank to maintain a homogeneous mixture. Alternatively, in other embodiments, the purified benzoic acid product and the alcohol are fed continuously to the agitated vessel containing the azeotropic agent and esterification catalyst. The water produced as a byproduct of the esterification reaction is removed as a vapor.

It is important that both a high conversion and selectivity is obtained for the esterification of the purified benzoic acid to benzoate esters. Thus, the esterification of purified benzoic acid typically has a conversion greater than 80%, 90%, 95%, 98%, or 99%. In addition to a high conversion, a high purity of the desired benzoic ester is also desired so fewer byproducts exist. The purity for the esterification of the purified benzoic acid is typically greater than 80%, 90%, 95%, 98%, or 99%.

The third embodiment of the current invention is a method for producing benzoate ester plasticizers comprising: contacting toluene in an oxidation vessel with a benzoic acid solvent, an oxygen containing gas, and an oxidation catalyst to produce a crude benzoic acid product; feeding the crude benzoic acid product to an esterification vessel where the crude benzoic acid product is contacted with an alcohol, an esterification catalyst, and an azeotropic agent to produce a crude benzoate ester plasticizer; washing the crude benzoate ester plasticizer with a caustic treatment comprising sodium carbonate and sodium hydroxide; and filtering the crude benzoate ester plasticizer with a filtration aid to produce a benzoate ester plasticizer with a reduced impurity profile.

It is understood that the descriptions outlining and teaching the method of producing a benzoate ester previously discussed, which can be used in any combination, apply equally well to the third embodiment of the invention additionally used to produce benzoate plasticizers.

The term “reduced impurity profile”, as used herein, refers to a benzoate ester plasticizer that has a higher total percentage weight of the final benzoate ester plasticizer compound. This “reduced impurity profile” is the result from the caustic treatment of the crude benzoate ester product; the filtration of the crude benzoate ester product; the washing the crude benzoate ester product; the decolorization or bleaching of the crude benzoate ester product, the strip-off of the crude benzoate ester product, or any combination thereof.

The caustic treatment of the crude benzoate plasticizer product is carried out at a temperature from 50 to 100° C. using a base at a concentration of 1 to 26 weight % in water. Examples of suitable bases include, but are not limited to, sodium carbonate, sodium hydroxide, sodium bicarbonate, potassium carbonate, potassium hydroxide, and potassium bicarbonate. In some embodiments, the base is a 26 weight % aqueous sodium carbonate solution or a 10 weight % sodium hydroxide aqueous solution. The crude benzoate ester plasticizer is washed with water, preferably at a pH greater than 7, to remove the water soluble impurities including the residual oxidation catalyst and/or the residual esterification catalyst.

The filtration and wash step is performed by running the crude benzoate ester plasticizer product through a pad of a porous filtration aid. Examples of porous filtration aides include, but are not limited to: celite diatomite comprising silicon dioxide (80 to 90 weight %), aluminum oxide (2 to 4 weight %), and iron oxide (0.5 to 2 weight %); perlite comprising silicon dioxide (70 to 75 weight %), aluminum oxide (12 to 15 weight %); cellulose; and activated carbon.

The decolorization or bleaching step is performed at a temperature from 50 to 100° C. using a bleaching agent and/or activated carbon. Typical bleaching agents can be used such as peroxide-based bleach (i.e. hydrogen peroxide, sodium percarbonate, sodium perborate) and chlorine-based bleaches (i.e. sodium hypochlorite and calcium hypochlorite). In some embodiments, the bleaching agent is hydrogen peroxide.

The strip-off step is used to remove volatile impurities. Examples of practicing the strip-off step may include a steam strip-off or a vacuum strip-off at 10 to 50 mmHg.

It is to be understood that variations and modifications can be made on the aforementioned structures and methods without departing from the concepts of the present device, and further it is to be understood that such concepts are intended to be covered by the following claims unless these claims by their language expressly state otherwise.

The above descriptions are considered that of the illustrated embodiments only. Modifications of the disclosed methods will occur to those skilled in the art and to those who use these methods. Therefore, it is understood that the embodiments shown in the drawings and described above is merely for illustrative purposes and not intended to limit the scope of these disclosed methods, which is defined by the following claims as interpreted according to the principles of patent law, including the Doctrine of Equivalents.

The term “Conversion”, as used herein, is defined as the moles of reagent reacted divided by moles of reagent fed.

The term “Yield”, as used herein, is defined as the moles of each reaction product species created per mole of reagent reacted. Yield calculations do not include unreacted reagent.

### Listing of Non-Limiting Embodiments

Embodiment A is a method for producing benzoate esters comprising contacting toluene in an oxidation vessel with a benzoic acid solvent, an oxygen containing gas, and an oxidation catalyst to produce a crude benzoic acid product; transferring the crude benzoic acid product to an esterification vessel where the crude benzoic acid product is contacted with an alcohol, an esterification catalyst, and an azeotropic agent to produce a crude benzoate ester product; and treating the crude benzoate ester product to produce a benzoate ester with a reduced impurity profile.

The method of Embodiment A, wherein the oxidation catalyst comprises cobalt acetate, cobalt hydroxide, cobalt carbonate, cobalt benzoate, manganese acetate, manganese carbonate, manganese benzoate, or combinations thereof.

The method of Embodiment A or Embodiment A with one or more of the intervening features wherein the esterification catalyst comprises tin, titanium, zinc, zirconium, or combinations thereof.

The method of Embodiment A or Embodiment A with one or more of the intervening features wherein the crude benzoic acid product comprises benzaldehyde, biphenyl, fluorenone, benzyl benzoate, phenoxymethyl benzoate, and 3,4-benzocoumarin.

The method of Embodiment A or Embodiment A with one or more of the intervening features further comprising washing the crude benzoate ester plasticizer with a caustic treatment comprising sodium carbonate and sodium hydroxide; and filtering the crude benzoate ester plasticizer with a porous filtration aid to produce a benzoate ester plasticizer with a reduced impurity profile.

The method of Embodiment A or Embodiment A with one or more of the intervening features wherein the alcohol comprises at least one of the following structures:

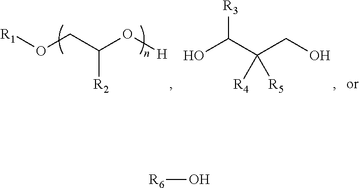

wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, R₅ is a hydrogen, methyl, or ethyl group, and R₆ is an alkyl group having 1 to 18 carbon atoms.

The method of Embodiment A or Embodiment A with one or more of the intervening features wherein the azeotropic agent comprises petroleum distillate, toluene, xylene, isooctane, or a combination thereof.

The method of Embodiment A or Embodiment A with one or more of the intervening features wherein the crude benzoic acid product is flashed to the esterification vessel.

The method of Embodiment A or Embodiment A with one or more of the intervening features wherein the benzoate ester has a yield of at least 80% and a purity of at least 80%.

Embodiment B is a method for producing benzoate esters comprising contacting toluene in an oxidation vessel with a benzoic acid solvent, an oxygen containing gas, and an oxidation catalyst to produce a crude benzoic acid product; flashing the crude benzoic acid product to give a purified benzoic acid; contacting the purified benzoic acid in an esterification vessel with an alcohol, an esterification catalyst, and an azeotropic agent to produce a crude benzoate ester product; and treating the crude benzoate ester product to produce a benzoate ester

The method of Embodiment B wherein the oxidation catalyst comprises cobalt acetate, cobalt hydroxide, cobalt carbonate, cobalt benzoate, manganese acetate, manganese carbonate, manganese benzoate, or a combination thereof.

The method of Embodiment B or Embodiment B with one or more of the intervening features wherein the esterification catalyst comprises tin, titanium, zinc, zirconium, or a combination thereof.

The method of Embodiment B or Embodiment B with one or more of the intervening features wherein the unpurified crude benzoic acid product comprises benzaldehyde, biphenyl, fluorenone, benzyl benzoate, phenoxymethyl benzoate, and 3,4-benzocoumarin.

The method of Embodiment B or Embodiment B with one or more of the intervening features wherein the alcohol comprises at least one of the following structures:

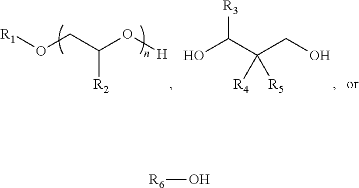

wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, and R₅ is a hydrogen, methyl, or ethyl group, and R₆ is an alkyl group having 1 to 18 carbon atoms.

The method of Embodiment B or Embodiment B with one or more of the intervening features wherein the benzoate ester is incorporated into a polymer material.

Embodiment C is a method for producing benzoate ester plasticizers comprising contacting toluene in an oxidation vessel with a benzoic acid solvent, an oxygen containing gas, and an oxidation catalyst to produce a crude benzoic acid product; feeding the crude benzoic acid product to an esterification vessel where the crude benzoic acid product is contacted with an alcohol, an esterification catalyst, and an azeotropic agent to produce a crude benzoate ester plasticizer; washing the crude benzoate ester plasticizer with a caustic treatment comprising sodium carbonate and sodium hydroxide; and filtering the crude benzoate ester plasticizer with a filtration aid to produce a benzoate ester plasticizer with a reduced impurity profile.

The method of Embodiment C wherein the oxidation catalyst comprises cobalt (II) acetate, manganese (II) acetate, and aqueous hydrobromic acid.

The method of Embodiment C or Embodiment C with one or more of the intervening features wherein the esterification catalyst comprises titanium (IV) isopropoxide.

The method of Embodiment C or Embodiment C with one or more of the intervening features wherein the alcohol comprises at least one of the following structures:

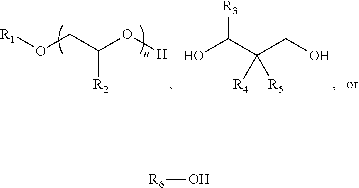

wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, and R₅ is a hydrogen, methyl, or ethyl group, and R₆ is an alkyl group having 1 to 18 carbon atoms.

The method of Embodiment C or Embodiment C with one or more of the intervening features wherein the benzoate ester is incorporated into a polymer material.

### EXAMPLES

### Materials

Aqueous hydrobromic acid (48.7%), cobalt (II) acetate tetrahydrate and manganese (II) acetate tetrahydrate were purchased from J. T. Baker. Benzoic acid and toluene were obtained from Aldrich, Alpha Aesar, Kalama, and KJ. All chemicals were used as received.

### Gas Chromatography Analysis

### GC Method 1

Method 1 was performed on the product mixtures resulting from the oxidation of toluene to make benzoic acid. These samples were analyzed by capillary gas chromatography using an Agilent model 7890B gas chromatograph equipped with a split injector and a flame ionization detector. The gas chromatograph was interfaced to an EZChrom Elite Chromatography Data System. The column used was a DB™-5 (5% phenyl-95% dimethylpolysiloxane column) fused silica capillary column having 30 meters×0.25 mm I.D., 0.25 micron film. The temperature profile used for the analysis was isothermal at 80° C. for 2 minutes, then programmed to 320° C. at a rate of 16° C./minute; the final 320° C. temperature was held for 15 minutes. The carrier gas was Helium with a constant 10 psi back pressure. A split injector set at 250° C. was used with a 50:1 split ratio. The detector used flame ionization at 340° C.

Two different sample preparation methods were developed to best determine all the components of interest. Quantitation for both methods was performed by internal standardization in order to provide weight percent concentrations of all components. The first method was used to determine the following components: toluene, benzaldehyde, phthalic anhydride, biphenyl, isomers of methyl biphenyl, benzophenone, 9H-fluoren-9-one, benzyl benzoate, benzil, phenoxymethyl benzoate, and 3,4-benzocoumarin. The samples were prepared by weighing 0.2 grams of sample together with 0.02 grams of decane (the internal standard), to an accuracy of 0.1 mg. The weighed sample was then diluted with 4 mL of acetone and vortexed to assure complete dissolution. The injection volume was 2 microliters.

The second method was used to determine the following components: benzoic acid, phthalic acid, isophthalic acid, biphenyl 2-carboxylic acid, terephthalic acid, biphenyl 3-carboxylic acid, biphenyl 4-carboxylic acid, 2-benzoylbenzoic acid, and 4-benzoylbenzoic acid. Both palmitic and stearic acid were seen in all samples, but quantitation of these components was not performed for this work. The samples were prepared by weighing 0.1 gram of sample with 0.02 grams dodecane (the internal standard), to an accuracy of 0.1 mg. The weighed sample was then diluted with 2 mL N,N-dimethylformamide (DMF) and vortexed to assure complete dissolution. Approximately 10 drops of the sample solution was derivatized with 750 microliters of N,O-bis(trimethylsilyl)trifluoroacetamide (BSTFA). The derivatized solution was heated at 80° C. for 10-15 minutes. Injection volume was 2 microliters.

### GC Method 2

Method 2 was performed on the product mixtures resulting from the esterification of benzoic acid to make benzoic esters such as diethylene glycol and dipropylene glycol benzoates. These samples were analyzed by capillary gas chromatography using an Agilent model 7890B gas chromatograph equipped with a split injector and a flame ionization detector. The gas chromatograph was interfaced to an EZChrom Elite Chromatography Data System. The column used was a DB™-1701 fused silica capillary column, 30 meters×0.25 mm I.D.×1.00 micron film. The temperature profile used for the analysis was isothermal at 80° C. for 1 minutes, then programmed to 150° C. at a rate of 15° C./minute. The temperature is held at 150° C. for 4 minutes, then programmed to 280° C. at 15° C./minute; the final temperature of 280° C. was held for 12 minutes. The carrier gas was hydrogen using a ramped flow mode with a flow program of 2 mL/minute for 17 minutes, then programmed to 4 mL/minute at a rate of 0.2 mL/minute, with a final flow of 2 mL/minute held for the remainder of the run. A split injector set at 250° C. was used with a 40:1 split ratio. The detector used flame ionization at 300° C.

The samples were prepared by adding 10 drops of samples to 3 mL of acetone. The injection volume was 1 microliters. Quantitation of the individual components was calculated using the area percent.

### GC Method 3

Method 3 was performed as a 45 minute method to analyze the high boiling impurities in the benzoic ester product mixtures such as brominated diethylene glycol benzoate. These samples were analyzed by capillary gas chromatography using an Agilent model 5890 gas chromatograph equipped with a split injector and a flame ionization detector. The gas chromatograph was interfaced to an EZChrom Elite Chromatography Data System. The column used was a DB™-5 fused silica capillary column, 30 meters×0.25 mm I.D.×0.25 micron film. The temperature profile used for the analysis was isothermal at 80° C. for 2 minutes, then programmed to 200° C. at a rate of 20° C./minute. The temperature was held at 200° C. for 2 minutes, then programmed to 280° C. at 20° C./minute; the final temperature of 280° C. was held for 31 minutes. The carrier gas was Helium with a constant 20 psi back pressure. A split injector set at 280° C. was used with a 40:1 split ratio. The detector used flame ionization at 320° C.

The samples were prepared by adding 2 drops of samples to 1.5 mL of acetone. The injection volume was 2 microliters. Quantitation of the individual components was calculated using the area percent.

### GC Method 4

Method 4 was performed for the analysis of other benzoate esters. These samples were analyzed by capillary gas chromatography using an Shimadzu GC-2010 plus gas chromatograph equipped with a split injector and a flame ionization detector. The column used was a DB™-1701 fused silica capillary column, 60 meters×0.25 mm I.D.×0.25 micron film. The temperature profile used for the analysis was isothermal at 80° C. for 1 minute, then programmed to 150° C. at a rate of 15° C./minute. The temperature was held at 150° C. for 4 minutes and then programmed to 280° C. at 15° C./minute, the temperature was held at 280° C. for 5 minutes, then programmed to 300° C. at 15° C./minute; the final temperature of 300° C. was held for 10.33 minutes. The carrier gas was Helium with a constant 10 psi back pressure. A split injector set at 300° C. was used with a 20:1 split ratio. The detector used flame ionization at 250° C.

The samples were prepared by adding 2 drops of samples to 1.0 mL of acetone. The injection volume was 2 microliters. Quantitation of the individual components was calculated using the area percent.

### UNIQUANT™ Analysis

UNIQUANT™ X-ray fluorescence (XRF) analysis was performed to measure the chemical composition of the benzoic acid compositions. This analysis was carried out on a PANalytical Axios Advanced X-ray Fluorescence instrument. The samples were run neat and no sample preparation was performed.

### Color Measurement

A Carver Press was used on a 40 mm plastic cup (Chemplex Plasticup, 39.7×6.4 mm) with the benzoic acid sample. The cup was then placed into a Nasco Whirl-Pak 4 oz plastic bag. A HunterLab Colorquest XE colorimeter using Hunterlab EasyQuest QC software in Reflectance Specular Included-Large Area View (RSIN-LAV) mode was used to measure the CIE L* a* b* and X Y Z values. The values obtained should be ±0.15 units on each scale of the stated values. Duplicate readings were recorded and the values were averaged to report.

### Synthesis of Crude Benzoic Acid

### Example 1

Benzoic acid (150.0 g, 1228.3 mmol), Co(OAc)₂.4H₂O (395 mg, 1.59 mmol), Mn(OAc)₂.4H₂O (35 mg, 0.14 mmol), 48.7 weight % aqueous hydrobromic acid (77 mg, 0.46 mmol) and water (6.0 g, 333 mmol) were charged to a 300 mL titanium autoclave equipped with a high pressure condenser, a baffle and an Isco pump. The autoclave was pressurized with approximately 50 psig of nitrogen and the mixture was heated to 160° C. in a closed system (i.e., with no gas flow) with stirring. At the desired reaction temperature, an air flow of 1200 sccm was introduced at the bottom of the solution and the reaction pressure was adjusted to 100 psig pressure. Toluene was then fed to the mixture at a rate of 0.35 mL/min via a high pressure Isco pump (the beginning of this feed was t=0 for the reaction time). The feed was stopped after 1 hour and the reaction continued for an additional 2 hours at the same air flow, temperature, and pressure conditions. After the reaction time was completed, the air flow was stopped and the autoclave was cooled to room temperature, depressurized and unloaded. The reaction conditions are given in Table 1. The solid product was analyzed by GC Method 1 and the results are given in Table 2.

### Examples 2-9

The reaction conditions disclosed in Example 1 were repeated in Examples 2-9 but the catalyst amounts and reaction conditions were varied as shown in Table 1. The solid product crude benzoic acid was analyzed for each Example using GC Method 1 and the results are given in Table 2.

### Example 10

This Example was conducted using ¹³C labeled Co(OAc)₂.4H₂O and ¹³C labeled Mn(OAc)₂.4H₂O as catalysts. Example 1 was repeated using the catalysts and reaction conditions given in Table 1. The results from this experiment are given in Table 2.

TABLE 1
Reaction conditions for the catalyzed air oxidation of toluene in benzoic acid solvent.^{a}

| | | | | | | | | toluene | |
|---|---|---|---|---|---|---|---|---|---|
| Exam- | [Co] | Co(OAc)₂•4H₂O | [Mn] | Mn(OAc)₂•4H₂O | [Br] | HBR | temp. | feed | air |
| ple | ppm | mg | ppm | mg | ppm | mg | ° C. | (mL/min) | (sccm) |
| 1 | 600 | 395 | 50 | 35 | 250 | 77 | 160 | 0.35 | 1200 |
| 2 | 300 | 190 | 30 | 20 | 250 | 77 | 160 | 0.35 | 1200 |
| 3 | 300 | 190 | 30 | 20 | 250 | 77 | 160 | 0.35 | 1200 |
| 4 | 300 | 190 | 30 | 20 | 250 | 77 | 160 | 0.35 | 600 |
| 5 | 300 | 190 | 30 | 20 | 250 | 77 | 160 | 0.35 | 1200 |
| 6 | 150 | 95 | 15 | 10 | 150 | 46 | 160 | 0.35 | 1200 |
| 7 | 75 | 48 | 8 | 5 | 75 | 23 | 160 | 0.35 | 1200 |
| 8 | 30 | 19 | 300 | 200 | 250 | 77 | 177 | 0.35 | 1200 |
| 9 | 100 | 66 | 200 | 139 | 250 | 77 | 180 | 0.35 | 1200 |
| 10 | 300 | b | 30 | c | 250 | 77 | 160 | 0.35 | 1200 |

^{a}P = 100 psig, Reaction time is 3 h except run 5. Reaction time for run 5 is 5 h.
b. ¹³C labeled Co(OAc)₂4H₂O was used.
c. ¹³C labeled Mn(OAc)₂4H₂O was used.

TABLE 2
Results of the catalyzed air oxidation of toluene in benzoic acid solvent.

| | | | weight % | | | | | | |
|---|---|---|---|---|---|---|---|---|---|
| Exam- | | % | | | | Benzyl | Phenoxymethyl | 3,4- | total |
| ple | b* | conversion | Benzaldehyde | Biphenyl | Fluoenone | Benzoate | benzoate | Benzocoumarin | biphenyls^{a} |
| 1 | 4.07 | 100 | ND | 0.004 | 0.002 | ND | 0.010 | 0.031 | 0.170 |
| 2 | 4.3 | 100 | 0.001 | 0.003 | ND | ND | 0.018 | 0.022 | 0.089 |
| 3 | 4.8 | 99.9 | 0.009 | 0.004 | 0.001 | ND | 0.020 | 0.036 | 0.122 |
| 4 | 4.3 | 99.8 | ND | 0.004 | 0.006 | ND | 0.023 | 0.021 | 0.273 |
| 5 | 6.6 | 98.7 | ND | 0.003 | 0.004 | ND | 0.009 | 0.006 | 0.104 |
| 6 | 3.5 | 72 | 0.339 | ND | ND | 0.097 | 0.011 | 0.001 | 0.036 |
| 7 | 3 | 79.3 | 0.292 | ND | ND | 0.084 | 0.008 | 0.001 | 0.010 |
| 8 | 14.34 | 97.2 | ND | 0.002 | 0.0002 | 0.0009 | 0.016 | 0.002 | 0.059 |
| 9 | 5.94 | 99.5 | ND | 0.004 | ND | ND | 0.018 | 0.018 | 0.084 |
| 10 | 3.27 | 99.4 | ND | 0.004 | ND | ND | 0.016 | 0.016 | 0.097 |

^{a}Total biphenyls is the sum of the weight % of biphenyl, biphenyl-2-carboxylic acid, biphenyl-3-carboxylic acid and biphenyl-4-carboxylic acid.

Catalyst Stock Solution Preparation for Examples 11-12:

Co(OAc)₂.4H2O (3.17 g, 12.74 mmol), Mn(OAc)₂.4H₂O (0.334 g, 1.36 mmol), 48.7 weight % aqueous hydrobromic acid (1.54 g, 9.26 mmol) and water (94.96 g) were mixed to make a stock solution.

### Example 11

Benzoic acid (80.0 g, 655.1 mmol) and 2.7 g of catalyst stock solution were charged to a 300 mL titanium autoclave equipped with a high pressure condenser, a baffle and an Isco pump. The autoclave was pressurized with approximately 50 psig of nitrogen and the mixture was heated to 160° C. in a closed system (i.e., with no gas flow) with stirring. At reaction temperature, an air flow of 600 sccm was introduced at the bottom of the solution and the reaction pressure was adjusted to 100 psig pressure. Toluene was fed to the mixture at a rate of 0.35 mL/min via a high pressure Isco pump (this is t=0 for the reaction time). The feed was stopped after 233 minutes. The reaction was continued for an additional 60 minutes. The catalyst solution was also fed with the toluene feed at a rate of 0.016 mL/min via a second high pressure Isco pump. The catalyst solution feed was stopped after 206 minutes. After the reaction time was completed, the air flow was stopped and the autoclave was cooled to room temperature, depressurized, and unloaded. The solid crude benzoic acid product was analyzed by GC Method 1 and is shown in Table 3.

### Example 12

Recycled crude benzoic acid from Example 11 (80.0 g, 655.1 mmol) and 3.9 g of catalyst stock solution were transferred to a 300 mL titanium autoclave equipped with a high pressure condenser, a baffle and an Isco pump. The autoclave was pressurized with approximately 50 psig of nitrogen and the mixture was heated to 160° C. in a closed system (i.e., with no gas flow) with stirring. At reaction temperature, an air flow of 600 sccm was introduced at the bottom of the solution and the reaction pressure was adjusted to 100 psig pressure. Toluene was fed to the mixture at a rate of 0.35 mL/min via a high pressure Isco pump (this is t=0 for the reaction time). The feed was stopped after 233 min. The reaction was continued for an additional 177 min. Catalyst solution was also fed with the toluene feed at a rate of 0.022 mL/min via a second high pressure Isco pump. The catalyst solution feed was stopped after 206 min. After the reaction time was completed, the air flow was stopped and the autoclave was cooled to room temperature, depressurized, and unloaded. The solid product was analyzed by GC Method 1 and is shown in Table 3.

### Example 13

A 300 mL Ti autoclave equipped with a high pressure condenser, a baffle and an Isco syringe pump was charged with 80 g of purified benzoic acid, cobaltous acetate tetrahydrate (1.90 g; 7.6 mmol), manganous acetate tetrahydrate (0.053 g; 0.2 mmol), and 0.67 g of 49% aqueous HBr (4.1 mmol). The autoclave was sealed and pressurized to 100 psig with air. The mixture was heated to 165° C. and stirred at 900 rpm. Air was fed to the autoclave at a rate of 1900 sccm, and toluene was fed through the Isco pump at a rate of 0.35 mL/min for 4 hours. The autoclave was cooled to room temperature, vented, and the crude benzoic acid was removed. The amount of crude benzoic acid that was recovered was 174 g. The crude material was ground with a mortar and transferred to a 500 mL round bottom flask where it was stored under vacuum (1 torr) until it reached constant weight (166 g). The yield of crude benzoic acid (minus the starting benzoic acid) recovered was calculated to be 91%. The solid product was analyzed by GC Method 1 and is shown in Table 3.

### Example 14

A 1 L Ti autoclave equipped with a high pressure condenser, a baffle and an Isco pump was charged with 166 g of recycled crude benzoic acid made in Example 13, cobaltous acetate tetrahydrate (2.95 g; 11.8 mmol), manganous acetate tetrahydrate (0.082 g; 0.3 mmol), and 1.04 g of 49% aqueous HBr (6.3 mmol). The autoclave was sealed and pressurized to 100 psig with air. The mixture was heated to 165° C. and stirred at 900 rpm. Air was fed to the autoclave at a rate of 1900 sccm, and toluene was fed through the Isco pump at a rate of 0.35 mL/min for 5 hours. The autoclave was cooled to room temperature, vented, and the crude benzoic acid was removed. The crude material was ground with a mortar and transferred to a 1000 mL round bottom flask where it was stored under vacuum (1 torr) until it reached constant weight (408 g). The yield of crude benzoic acid recovered was calculated to be 75%. The crude benzoic acid was returned to the 1 L autoclave along with cobaltous acetate tetrahydrate (2.54 g; 10.2 mmol), manganous acetate tetrahydrate (0.071 g; 0.3 mmol), and 0.80 g of 49% aqueous HBr (4.8 mmol), and the procedure above was followed except that 1700 sccm of air and 0.48 mL/min of toluene were fed over 4 h. The product was worked up as described to give a final weight of 547 g of crude benzoic acid (96% yield). The solid product was analyzed by GC Method 1 and is shown in Table 3.

TABLE 3
Results of the catalyzed air oxidation of toluene in benzoic acid solvent in a recycled benzoic acid solvent.

| | | | weight % | | | | | | |
|---|---|---|---|---|---|---|---|---|---|
| Exam- | | % | | | | Benzyl | Phenoxy- | 3,4-Benzo- | total |
| ple | b* | conversion | Benzaldehyde | Biphenyl | Fluoenone | Benzoate | methylbenzoate | coumarin | biphenyls^{a} |
| 11 | 5.91 | 61.2 | 1.930 | 0.012 | 0.0030 | 0.403 | 0.027 | 0.006 | 0.222 |
| 12 | 17.04 | 100 | 0.002 | 0.012 | 0.0008 | 0.004 | 0.004 | 0.001 | 0.234 |
| 13 | 31.57 | 99.9 | 0.002 | 0.013 | 0.0036 | 0.012 | 0.054 | 0.002 | 0.282 |
| 14 | — | — | 0.495 | 0.083 | 0.020 | 0.465 | 0.019 | 0.3325 | 0.926 |

^{a}Total biphenyls is the sum of the wt. % of biphenyl, biphenyl-2-carboxylic acid, biphenyl-3-carboxylic acid and biphenyl-4-carboxylic acid

### Synthesis of Benzoate Esters

Various grades of benzoic acid were used to produce dibenzoates in the following examples. Commercial benzoic acid was purchased from Kalama or Alpha Aesar and used without further purification. The crude benzoic acid was obtained from manufacturing or lab processes for making benzoic acid from toluene. Crude benzoic acid from lab processes was purified by flash distillation to deliver lab flash benzoic acid and pot residue. A sample of each type of benzoic acid used for these studies was analyzed using GC Method 1 and the results are given in Table 4. Benzoic acid A was from Alpha Aesar, benzoic acid B was from Kalama, benzoic acid C was from a crude version from KJ, benzoic acid D was a lab crude (Example 14), benzoic acid E was lab flashed version, and benzoic acid F was the pot residue left after the flash.

TABLE 4
Benzoic Acid Analysis (GC Method 1)

| | | | | | E | F |
|---|---|---|---|---|---|---|
| | A | B | C | D | (Lab | (pot residue, |
| Benzoic Acid | (Alpha Aesar) | (Kalama) | (KJ crude) | (Lab crude) | flash) | lab flash) |
| Toluene | ND | ND | 0.473 | 0.765 | 0.062 | ND |
| Benzaldehyde | ND | ND | 0.681 | 0.495 | 0.336 | 0.010 |
| Phthalic Anhydride | ND | 0.157 | 0.226 | 0.229 | 0.866 | 0.875 |
| Biphenyl | ND | ND | 0.947 | 0.083 | 0.086 | 0.002 |
| Benzophenone | ND | ND | 0.015 | 0.012 | 0.010 | 0.041 |
| Fluorenone | ND | ND | 0.016 | 0.020 | 0.006 | 0.143 |
| Benzyl Benzoate | ND | ND | 1.132 | 0.465 | 0.192 | 2.358 |
| Benzil | ND | ND | ND | 0.002 | 0.001 | 0.011 |
| Phenoxymethyl | ND | ND | 0.037 | 0.019 | 0.003 | 0.091 |
| benzoate | | | | | | |
| 3,4- | 0.001 | 0.001 | 0.321 | 0.325 | 0.028 | 2.421 |
| Benzocoumarin | | | | | | |
| Benzoic Acid | 119.640 | 99.560 | 94.320 | 96.366 | 98.951 | 74.501 |
| Phthalic acid | 0.001 | 0.0644 | 0.230 | 0.143 | 0.042 | 0.065 |
| Isophthalic acid | 0.0033 | ND | 0.053 | 0.028 | ND | 0.089 |
| Biphenyl-2- | ND | ND | 0.040 | 0.055 | 0.010 | 0.323 |
| carboxylic acid | | | | | | |
| TPA | 0.025 | ND | 0.091 | 0.030 | 0.013 | 0.121 |
| Biphenyl-3- | 0.003 | ND | 0.368 | 0.347 | 0.048 | 2.386 |
| carboxylic acid | | | | | | |
| Biphenyl-4- | 0.007 | ND | 0.535 | 0.441 | 0.077 | 3.276 |
| carboxylic acid | | | | | | |
| 2-Benzoylbenzoic | ND | ND | 0.065 | ND | ND | ND |
| acid | | | | | | |
| 4-Benzoylbenzoic | ND | ND | 0.139 | ND | ND | ND |
| acid | | | | | | |

### Example 15

A two liter, 3-necked, round-bottomed flask with a bottom sampling port was equipped with an overhead mechanical stirrer, nitrogen inlet, temperature probe, and a decanter equipped with both a thermocouple and condenser. A dry flask was charged with benzoic acid B (457.0 g, 3.74 mol), diethylene glycol (176.6 g, 1.66 mol), dipropylene glycol (56.1 g, 0.41 mol), zirconium carbonate (0.32 g, 1.03 mmol), stannous oxalate (0.16 g, 0.77 mmol) and ISOPAR C (40 g). Sub-surfaced nitrogen flow and cooling water was initiated. The resulting mixture was gradually heated to 180° C. over a 4.5 hour period with the continuous removal of water. The resulting mixture was heated at 180° C. for an additional 4 hours. The amount of water in the decanter was recorded at the end of the reaction time (68.02 g, 100% yield).

Once the mixture was cooled to 80° C., saturated sodium carbonate solution was added. The resulting mixture was stirred for 30 minutes and then the aqueous layer was removed. The organic layer was washed successively with saturated sodium carbonate solution and water. The crude product was bleached with hydrogen peroxide at 80° C., and the volatiles were removed by steam strip-off at 50 mmHg, and the benzoate ester product was obtained as pale yellow liquid (586 g, 94% material balance). The benzoate ester product was analyzed by GC Method 2 and GC Method 3 and was determined to have an overall purity of 98.5% (88.4% dibenzoate and 10.1% monobenzoate). The results from the GC analysis are shown in Tables 5 and 6, respectively.

TABLE 5
Analysis of selected benzoate ester products
using GC Method 2 (area percentage)

| Example | 15 | 25 | 28 | 29 | 31 |
|---|---|---|---|---|---|
| Sample Info | From | From | From | From | From |
| | benzoic | benzoic | benzoic | benzoic | benzoic |
| | acid | acid | acid | acid | acid |
| | B | B | C | D | E |
| Material balance (%) | 94 | 92 | 83 | 71 | 78.5 |
| Color (APHA) | 100 | 100 | 500 | 500 | 500 |
| benzaldehyde | 0.000 | 0.000 | 0.321 | 0.000 | 0.000 |
| benzyl alcohol | 0.000 | 0.002 | 0.070 | 0.000 | 0.000 |
| biphenyl/methyl | 0.266 | 0.092 | 1.290 | 0.097 | 0.127 |
| biphenyl | | | | | |
| benzophenone | 0.000 | 0.000 | 0.018 | 0.000 | 0.000 |
| benzyl benzoate | 0.000 | 0.000 | 0.000 | 0.000 | 0.261 |
| benzocoumarin/ | 0.000 | 0.000 | 0.364 | 0.000 | 0.000 |
| fluorenone | | | | | |
| Propyleneoxy propyl | 0.065 | 0.011 | 0.008 | 0.000 | 0.002 |
| benzoate | | | | | |
| diethylene glycol | 7.545 | 2.584 | 6.319 | 6.687 | 3.722 |
| monobenzoate | | | | | |
| dipropylene glycol | 2.560 | 4.020 | 2.478 | 2.880 | 2.744 |
| monobenzoate | | | | | |
| propylene glycol | 0.060 | 0.045 | 0.065 | 0.015 | 0.047 |
| dibenzoate | | | | | |
| ethylene glycol | 0.490 | 0.501 | 0.491 | 0.232 | 1.130 |
| dibenzoate | | | | | |
| dipropylene glycol | 16.577 | 15.873 | 15.568 | 16.576 | 14.976 |
| dibenzoate | | | | | |
| diethylene glycol | 71.879 | 76.727 | 72.220 | 71.352 | 76.504 |
| dibenzoate | | | | | |

TABLE 6
Analysis of selected benzoate ester products
using GC Method 3 (area percentage)

| Example | 15 | 25 | 28 | 29 | 31 |
|---|---|---|---|---|---|
| Sample Info | From | From | From | From | From |
| | benzoic | benzoic | benzoic | benzoic | benzoic |
| | acid | aicd | acid | acid | acid |
| Biphenyl/methyl | 0.000 | 0.000 | 1.267 | 0.108 | 0.121 |
| biphenyl | | | | | |
| benzophenone | 0.023 | 0.000 | 0.000 | 0.099 | 0.000 |
| diethylene glycol | 6.616 | 2.741 | 3.168 | 5.564 | 2.472 |
| monobenzoate | | | | | |
| dipropylene glycol | 3.428 | 4.227 | 3.306 | 3.450 | 4.731 |
| monobenzoate | | | | | |
| benzyl benzoate | 0.085 | 0.090 | 2.202 | 0.290 | 0.300 |
| diethylene glycol | 0.000 | 0.000 | 0.000 | 0.886 | 0.000 |
| benzoate/acetate | | | | | |
| benzocoumarin | 0.000 | 0.000 | 0.372 | 0.704 | 0.026 |
| ethylene glycol | 0.610 | 0.605 | 0.564 | 0.273 | 1.308 |
| dibenzoate/ | | | | | |
| propylene glycol | | | | | |
| dibenzoate | | | | | |
| diethylene glycol | 87.763 | 92.113 | 86.487 | 84.924 | 90.075 |
| dibenzoate/ | | | | | |
| dipropylene glycol | | | | | |
| dibenzoate | | | | | |
| brominated | 0.022 | 0.022 | 0.000 | 0.173 | 0.073 |
| diethylene glycol | | | | | |
| dibenzoate | | | | | |
| unidentified high | 0.000 | 0.000 | 1.111 | 2.276 | 0.064 |
| boiler impurities | | | | | |

### Examples 16-21

Examples 16-21 used the same procedure as Example 1 but used benzoic acid B in Example 16, benzoic acid A in Examples 17-21, in addition to using different batches of zirconium carbonate and/or stannous oxalate. The amount of water in the decanter was recorded as follows: Example 16, 42.76 g. 63% yield, Example 17, 41.75 g, 62% yield; Example 18, 48.15 g, 71% yield; Example 19, 47.52 g, 70% yield; Example 20, 46.95 g, 70% yield; Example 21, 47.91 g, 71 yield.

Examples 16-21 were not able to reproduce the high yield seen in Example 15 due to the use of different batches of zirconium and/or stannous oxalate.

### Example 22

Example 22 used the same procedure as Example 15 but used benzoic acid A zirconium carbonate (0.64 g, 2.06 mmol) and stannous oxalate (0.32 grams, 1.54 mmol) and gradually heated to 200° C. over a 4.0 hour period with the continuous removal of water through a decanter. The reaction mixture was stirred at 200° C. for an additional 8.5 hours. The amount of water in the decanter was recorded (65.72 g, 97% yield).

### Example 23

Example 23 used the same procedure as Example 22 but used benzoic acid A (482.6 g, 3.95 mol) and the reaction was gradually heated to 200° C. over a 3.5 hour period with the continuous removal of water through a decanter. The reaction mixture was stirred at 200° C. for additional 4.5 hour. The amount of water in the decanter was recorded (64.32 g, 93% yield).

### Example 24

Example 24 used the same procedure as Example 23 but used benzoic acid B (482.6 g, 3.95 mol) zirconium carbonate (1.60 g, 5.18 mmol) and stannous oxalate (0.80 grams, 3.86 mmol). The reaction was carried out for 9 hours. The amount of water in the decanter was recorded (67.57 g, 95% yield).

### Example 25

Example 24 was repeated but the reaction was stirred for 7 hours. The amount of water in the decanter was recorded (67.76 g, 95% yield). After the mixture was cooled to 80° C., saturated sodium carbonate solution was added. The resulting mixture was stirred for 30 minutes. Aqueous layer was removed. The organic layer was washed successively with saturated sodium carbonate solution and water. The crude product was then bleached with hydrogen peroxide at 80° C. The volatiles were removed by steam strip-off at 50 mmHg and the benzoate ester product was obtained as light yellow liquid (590 g, 92% material balance) with an overall purity of 99.2% (92.6% dibenzoate and 6.6% monobenzoate). The benzoate ester product was analyzed by GC Method 2 and GC Method 3, the results are shown in Tables 5 and 6, respectively.

### Example 26

Example 26 used the same procedure as Example 16 but used benzoic acid C (457 g, 3.74 mol). After 6.5 hours, additional benzoic acid C (35 g, 0.28 mol) was added and the resulting mixture was refluxed for another 19.5 hours. The total amount of water collected in the decanter was 61.42 g (85% yield).

After the mixture was cooled to 80° C., saturated sodium carbonate solution was added. The resulting mixture was stirred for 30 minutes. The crude material was cooled to ambient temperature and filtered through a pad of celite to remove the solid impurities. The filtrate was then washed successively with a saturated sodium carbonate solution and water. The crude product was then bleached with hydrogen peroxide at 80° C. The volatiles were removed by steam strip-off at 50 mmHg and the benzoate ester product was obtained as orangey brown liquid (502.03 g, 77% material balance) with an overall purity of 97.2% (80.9% dibenzoate and 16.3% monobenzoate).

### Example 27

Example 27 used the same procedure as Example 16 but used benzoic acid C. The reaction was stopped at 7.75 hours. The amount of water in the decanter was recorded (52.33 g, 48% yield).

### Example 28

Example 28 used the same procedure as Example 24, optimized reaction conditions, and was repeated using benzoic acid C with an additional charge of benzoic acid C (20 g, 0.16 mol) after 5 hours. The reaction was stopped after 10 hours. The reaction mixture was then cooled to 80° C. and saturated sodium carbonate solution was added. The resulting mixture was stirred for 30 minutes. The crude material was then cooled to ambient temperature and filtered through a pad of celite to remove any solid impurities. The filtrate was successively washed with saturated sodium carbonate solution and water and the crude product was then bleached with hydrogen peroxide at 80° C. The volatiles were removed by steam strip-off at 50 mmHg. The benzoate ester product was obtained as orangey brown liquid (546.52 g, 83% material) with an overall purity of 96.5% (87.7% dibenzoate and 8.8% monobenzoate). The benzoate ester product was analyzed by GC Method 2 and GC Method 3, the results are shown in Tables 5 and 6, respectively.

### Example 29

Example 29 used the same procedure as Example 16 but used benzoic acid D containing 1.5 weight % water. At 7.5 hours, 68.8 g water was collected (57% yield after counting off the amount of water in the benzoic acid). The reaction was continued for an additional 16.5 hours before the pot was cooled to ambient temperature. Zirconium carbonate (0.64 g, 2.06 mmol) and stannous oxalate (0.32 grams, 1.54 mmol) were charged. The resulting mixture was stirred for an additional 7.5 hours.

After the mixture was cooled to 80° C., saturated sodium carbonate solution was added and the resulting mixture was stirred for 30 minutes. The crude material was cooled to ambient temperature and filtered through a pad of celite to remove the solid impurities. The filtrate was washed successively with saturated sodium carbonate solution and water. The crude product was bleached with hydrogen peroxide at 80° C. and the volatiles were removed by steam strip-off at 50 mmHg. The benzoate ester product was obtained as orangey brown liquid (441.64 g, 71% material balance) with an overall purity of 97.4% (87.9% dibenzoate and 9.5% monobenzoate). The benzoate ester product was analyzed by GC Method 2 and GC Method 3, the results are shown in Tables 5 and 6, respectively.

### Example 30

A single-stage flash distillation of benzoic acid C was carried out over a period of about 8 hours at a vacuum between 1.5 and 3.2 torr at a temperature between 113 and 128° C. 401 g benzoic acid (flashed benzoic acid) was obtained as pale yellow solid.

Example 30 used the same procedure as Example 16 but used the flashed benzoic acid (401.20 g, 3.28 mol), diethylene glycol (155.0 g, 1.46 mol), dipropylene glycol (49.4 g, 0.36 mol), zirconium carbonate (0.64 g, 2.06 mmol) and stannous oxalate (0.32 grams, 1.54 mmol). After 5 hours, additional zirconium carbonate (0.64 g, 2.06 mmol) and stannous oxalate (0.32 grams, 1.54 mmol) were added when the reaction mixture was cooled to 50° C. The mixture was stirred for an additional 15 hours at 180° C. The amount of water in the decanter was recorded (51.71 g, 88% yield).

After the mixture was cooled to 80° C., saturated sodium carbonate solution was added. The resulting mixture was stirred for 30 minutes. The aqueous layer was removed. The organic layer was washed successively with saturated sodium carbonate solution and water. The crude product was bleached with hydrogen peroxide at 80° C. The volatiles were removed by steam strip-off at 50 mmHg, and the benzoate ester product was obtained as pale yellow liquid (426.07 g, 78% material balance) with an overall purity of 93.8% (84.4% dibenzoate and 9.4% monobenzoate).

### Example 31

A single-stage flash distillation of benzoic acid D was carried out over a period of about 8 hours at a vacuum between 58.8 and 59.9 torr at a temperature between 169.9 and 178.1° C. 800.6 g benzoic acid E was obtained as pale yellow solid.

Example 24 (optimized reaction conditions) was repeated using benzoic acid E (386.0 g, 3.16 mol), diethylene glycol (141.28 g, 1.33 mol), dipropylene glycol (44.89 g, 0.33 mol), zirconium carbonate (1.28 g, 4.12 mmol) and stannous oxalate (4.12 mmol). The reaction was refluxed for 7 hours at 200° C. and 54.6 g water was collected in the decanter (95.8% yield). After the mixture was cooled to 80° C., saturated sodium carbonate solution was added and the resulting mixture was stirred for 30 minutes. The crude material was cooled to ambient temperature and filtered through a pad of celite to remove any solid impurities. The filtrate was then washed successively with saturated sodium carbonate solution and water. The crude product was then bleached with hydrogen peroxide at 80° C. and the volatiles were removed by steam strip-off at 50 mmHg. Benzoate ester product was obtained as light brown liquid (413.4 g, 78.5% material) with an overall purity of 97.8% (91.4% dibenzoate and 6.4% monobenzoate). The benzoate ester product was analyzed by GC Method 2 and GC Method 3, the results are shown in Tables 5 and 6, respectively.

### Examples 32-34

Using the procedure of Example 16, spiking experiments were carried out with the addition of sodium bromide (0.394 g, 3.82 mmol), cobalt (II) acetate tetrahydrate (0.954 g, 3.84 mmol) and manganese carbonate (0.47 g, 4.08 mmol) respectively. The amount of water in the decanter was recorded as follows: Example 32: 46.77 g, 65% yield; Example 33: 43.1 g, 64% yield; Example 34: 37.19 g, 55 yield.

### Example 35

Example 35 used the same procedure as Example 33 but used zirconium carbonate (3.2 g, 10.3 mmol), stannous oxalate (1.6 grams, 7.7 mmol) and cobalt (II) acetate tetrahydrate (0.954 g, 3.84 mmol). The reaction was stopped at 10 hours. The amount of water in the decanter was recorded (67.54 g, 100% yield).

### Example 36

Example 36 used the same procedure as Example 16 but used stannous acetate (0.18 g, 0.76 mmol) instead of stannous oxalate for 8 hours. The amount of water in the decanter was recorded (41.29 g, 61% yield).

### Example 37

A dry 100-milliliter round-bottomed flask was equipped with Dean-Stark trap, nitrogen inlet, temperature probe and condenser. The flask was charged with stearyl alcohol (27.0 g, 0.1 mol), benzoic acid C (14.6 g, 0.12 mol) and toluene (10 mL). Titanium tetraisopropoxide (0.568 g, 2.0 mmol) was added. The resulting mixture was brought to reflux with continuous removal of water for 16 hours.

After the mixture was cooled to 80° C., 10% sodium hydroxide aqueous solution was added and stirred for 30 minutes. The resulting mixture was cooled to ambient temperature and filtered through a pad of celite. The filtrate was washed successively with 10% sodium hydroxide solution and dried with a brine solution followed by sodium sulfate and then dried over sodium sulfate. The volatiles were removed by strip-off under reduced pressure. Stearyl benzoate was obtained as brown liquid and analyzed using GC Method 4 (37.2 g, 99% yield, 96% purity).

### Example 38

According to procedure of Example 37, a mixture of ethylene glycol 2-ethylhexyl ether and diethylene glycol 2-ethylhexyl ether (85/15, 34.8 g, 0.2 mol), benzoic acid (29.3 g, 0.24 mol) and toluene (20 mL) were charged to 200-milliliter round-bottomed flask. Titanium tetraisopropoxide (1.13 g, 4.0 mmol) was added. The reaction was stopped at 8 hours. A mixture of 2-((2-ethylhexyl)oxy)ethyl benzoate and 2-(2-((2-ethylhexyl)oxy)ethoxy)ethyl benzoate was obtained as yellow liquid (46.3 g, 85% yield, 91% purity). The benzoate ester product was analyzed by GC Method 4, the results from the GC analysis are shown in Table 7.

### Example 39

According to procedure of Example 37, diethylene glycol monomethyl ether (24.0 g, 0.2 mol) and benzoic acid (29.3 g, 0.24 mol) were used. The reaction was stopped at 17 hours. 2-(2-methoxyethoxy)ethyl benzoate was obtained as brown liquid (40.8 g, 91% yield, 93% purity). The benzoate ester product was analyzed by GC Method 4, the results from the GC analysis are shown in Table 7.

### Example 40

According to procedure of Example 37, 1,4-cyclohexanedimethanol (28.8 g, 0.2 mol) and benzoic acid (58.6 g, 0.48 mol) were used. The reaction was stopped at 17 hours. Crystallization of crude product from hot ethyl acetate delivered 36.7 g off-white crystalline product (99% purity). Concentration of mother liquid delivered 27.8 g yellow solid (93% purity). Overall, cyclohexane-1,4-diylbis(methylene)dibenzoate was obtained in 92% yield. The benzoate ester product was analyzed by GC Method 4, the results from the GC analysis are shown in Table 7.

### Example 41

According to procedure of Example 27, 2,2,4-trimethylpentane-1,3-diol (14.2 g, 0.097 mol), benzoic acid (29.6 g, 0.24 mol) and stannous oxalate (0.40 g, 1.93 mmol) were used. The reaction was stopped at 16 hours. The product was obtained in 80% conversion as a mixture, which contained 19.1% 2,2,4-trimethylpentane-1,3-diol, 55.4% 3-hydroxy-2,2,4-trimethylpentyl benzoate and 14.6% 2,2,4-trimethylpentane-1,3-diyl dibenzoate. The benzoate ester product was analyzed by GC Method 4, the results from the GC analysis are shown in Table 7.

TABLE 7
Esterification Reaction Products

| GC Method #4 | | | | | | | | | |
|---|---|---|---|---|---|---|---|---|---|
| Exam- | | | | | | Temp | Time | | Purity |
| ple | Raw material | BA/ROH | Cat | Cat % | azeotrope | (° C.) | (h) | Yield (%) | (%) |
| 37 | stearyl alcohol | 1.20 | TIPT | 2 | Toluene | 115 | 16 | 99 | 96 |
| 38 | EEH/DEEH (85/15) | 1.20 | TIPT | 2 | Toluene | 115 | 8 | 85 | 91 |
| 39 | DM | 1.20 | TIPT | 2 | Toluene | 115 | 17 | 91 | 93 |
| 40 | CHDM | 2.40 | TIPT | 2 | Toluene | 115 | 17 | 92 | 96 |
| 41 | TMPD | 2.50 | Tin | 2 | Toluene | 115 | 16 | 80** | 70 |
| | | | oxalate | | | | | | |

* Yield % was calculated based on alcohol.
**Conversion % was calculated based on alcohol.

### UNIQUANT™ or Semi-Quantitative Analysis

Semi-quantitative analysis of the residual metal levels in the benzoic ester products was carried out using a X-Ray Fluorescence (XRF) spectrometer. For Examples 15, 26, 28, 29, 31, and 38, the zirconium catalyst was mostly removed during the sodium carbonate wash, while celite filtration efficiently removed the manganese, cobalt and tin catalyst. Comparable levels of metals were observed in the final benzoate ester prepared from unrefined benzoic acid and vendor benzoic acid. Similarly, high levels of metals in the solid filtrate were observed. The results from this UNIQUANT™ or semi-quantitative analysis are shown in Table 8. All values presented in the UNIQUANT™ or semi-quantitative report are listed as weight %. “ND” means that the concentration is less than 1 mg/kg, which is 0.0001 weight %, or not detected at all. The “<3e” notation means that the detected weight % is less than 3 standard errors.

TABLE 8
UNIQUANT ™ or semi-quantitative analysis of metal levels

| Exam- | Sample | | | | | | | | | | | | | |
|---|---|---|---|---|---|---|---|---|---|---|---|---|---|---|
| ple | Info | Na | Mg | Al | Si | K | Ca | Ti | Mn | Fe | Co | Zn | Zr | Sn |
| 28 | Aqueous, | 6.94 | ND | 0.0268 | 0.0077 | 0.0025 | 0.00042 | 0.00015 | 0.001 | <3e | <3e | <3e | 0.262 | <3e |
| | NaCO3 | | | | | | | | | | | | | |
| | wash | | | | | | | | | | | | | |
| 28 | Aq, 2nd | 5.04 | ND | 0.0041 | 0.0113 | 0.0004 | 0.002 | 0.00011 | 0.0055 | <3e | 0.00089 | <3e | 0.0102 | <3e |
| | Na2CO3 | | | | | | | | | | | | | |
| | wash | | | | | | | | | | | | | |
| 28 | 1st water | 0.062 | ND | ND | 0.0077 | 0.00038 | 0.0013 | ND | <3e | <3e | <3e | ND | 0.00075 | ND |
| | wash | | | | | | | | | | | | | |
| 28 | Solid | 1.46 | 0.253 | 0.641 | 3.54 | 0.351 | 0.697 | ND | 0.678 | 0.0076 | 0.039 | 0.651 | 0.0379 | 0.191 |
| | filtrate | | | | | | | | | | | | | |
| | on celite | | | | | | | | | | | | | |
| 28 | 2nd water | <3e | ND | ND | <3e | <3e | 0.0043 | 0.0001 | <3e | <3e | <3e | 0.00042 | <3e | ND |
| | wash | | | | | | | | | | | | | |
| 15 | Final | <3e | ND | ND | 0.0081 | ND | 0.0004 | ND | <3e | 0.00062 | ND | 0.0011 | 0.0181 | 0.0163 |
| | product | | | | | | | | | | | | | |
| 26 | Final | <3e | <3e | ND | 0.0109 | ND | 0.00025 | 0.0001 | 0.00045 | <3e | <3e | 0.00037 | <3e | ND |
| | product | | | | | | | | | | | | | |
| 29 | Final | <3e | ND | 0.0145 | 0.0165 | ND | 0.00022 | 0.00014 | <3e | <3e | ND | <3e | <3e | <3e |
| | product | | | | | | | | | | | | | |
| 31 | Final | ND | ND | <3e | 0.0039 | ND | 0.00011 | ND | <3e | <3e | <3e | <3e | ND | ND |
| | product | | | | | | | | | | | | | |
| 38 | Solid | 1.26 | 0.0935 | 0.263 | 16.34 | 0.0443 | 2.56 | 2.08 | 0.431 | 0.113 | 0.0286 | 0.0179 | 0.0027 | <3e |
| | filtrate | | | | | | | | | | | | | |
| | on celite | | | | | | | | | | | | | |
| 38 | Aqueous, | 5.89 | ND | 0.0141 | 0.0307 | 0.0012 | 0.0128 | 0.0084 | 0.0017 | <3e | <3e | 0.00061 | <3e | <3e |
| | NaOH | | | | | | | | | | | | | |
| | wash | | | | | | | | | | | | | |

All values reported in weight %
ND—Below detection limit or none detected
<3e—Detected weight % is less than 3 standard errors

It is not intended that the scope of the invention is to be limited by the Examples described and illustrated above, but instead it is intended the scope of the invention will be determined by the appended claims and their equivalents.

We claim:

1. A method for producing benzoate esters comprising:
contacting toluene in an oxidation vessel with a benzoic acid solvent, an oxygen containing gas, and an oxidation catalyst to produce a crude benzoic acid product;
transferring the crude benzoic acid product to an esterification vessel where the crude benzoic acid product is contacted with an alcohol, an esterification catalyst, and an azeotropic agent to produce a crude benzoate ester product; and
treating the crude benzoate ester product to produce a benzoate ester with a reduced impurity profile.

2. The method according to claim 1, wherein the oxidation catalyst comprises cobalt acetate, cobalt hydroxide, cobalt carbonate, cobalt benzoate, manganese acetate, manganese carbonate, manganese benzoate, or combinations thereof.

3. The method according to claim 1, wherein the esterification catalyst comprises tin, titanium, zinc, zirconium, or combinations thereof.

4. The method according to claim 1, wherein the crude benzoic acid product comprises benzaldehyde, biphenyl, fluorenone, benzyl benzoate, phenoxymethyl benzoate, and 3,4-benzocoumarin.

5. The method according to claim 1 further comprising:
washing the crude benzoate ester plasticizer with a caustic treatment comprising sodium carbonate and sodium hydroxide; and
filtering the crude benzoate ester plasticizer with a porous filtration aid to produce a benzoate ester plasticizer with a reduced impurity profile.

6. The method according to claim 1, wherein the alcohol comprises at least one of the following structures: 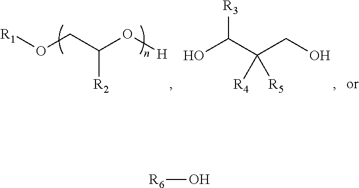
wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, R₅ is a hydrogen, methyl, or ethyl group, and R₆ is an alkyl group having 1 to 18 carbon atoms.

7. The method according to claim 1, wherein the azeotropic agent comprises petroleum distillate, toluene, xylene, isooctane, or a combination thereof.

8. The method according to claim 1, wherein the crude benzoic acid product is flashed to the esterification vessel.

9. The method according to claim 1, wherein the benzoate ester has a yield of at least 80% and a purity of at least 80%.

10. A method for producing benzoate esters comprising:
contacting toluene in an oxidation vessel with a benzoic acid solvent, an oxygen containing gas, and an oxidation catalyst to produce a crude benzoic acid product;
flashing the crude benzoic acid product to give a purified benzoic acid;
contacting the purified benzoic acid in an esterification vessel with an alcohol, an esterification catalyst, and an azeotropic agent to produce a crude benzoate ester product; and
treating the crude benzoate ester product to produce a benzoate ester.

11. The method according to claim 10, wherein the oxidation catalyst comprises cobalt acetate, cobalt hydroxide, cobalt carbonate, cobalt benzoate, manganese acetate, manganese carbonate, manganese benzoate, or a combination thereof.

12. The method according to claim 10, wherein the esterification catalyst comprises tin, titanium, zinc, zirconium, or a combination thereof.

13. The method according to claim 10, wherein the unpurified crude benzoic acid product comprises benzaldehyde, biphenyl, fluorenone, benzyl benzoate, phenoxymethyl benzoate, and 3,4-benzocoumarin.

14. The method according to claim 10, wherein the alcohol comprises at least one of the following structures: 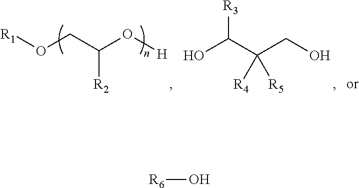
wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, and R₅ is a hydrogen, methyl, or ethyl group, and R₆ is an alkyl group having 1 to 18 carbon atoms.

15. The method according to claim 10, wherein the benzoate ester is incorporated into a polymer material.

16. A method for producing benzoate ester plasticizers comprising:
contacting toluene in an oxidation vessel with a benzoic acid solvent, an oxygen containing gas, and an oxidation catalyst to produce a crude benzoic acid product;
feeding the crude benzoic acid product to an esterification vessel where the crude benzoic acid product is contacted with an alcohol, an esterification catalyst, and an azeotropic agent to produce a crude benzoate ester plasticizer;
washing the crude benzoate ester plasticizer with a caustic treatment comprising sodium carbonate and sodium hydroxide; and
filtering the crude benzoate ester plasticizer with a filtration aid to produce a benzoate ester plasticizer with a reduced impurity profile.

17. The method according to claim 10, wherein the oxidation catalyst comprises cobalt (II) acetate, manganese (II) acetate, and aqueous hydrobromic acid.

18. The method according to claim 10, wherein the esterification catalyst comprises titanium (IV) isopropoxide.

19. The method according to claim 10, wherein the alcohol comprises at least one of the following structures: 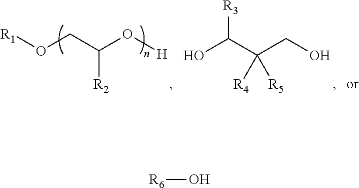
wherein R₁ is selected from the group consisting of hydrogen and an alkyl group having 1 to 8 carbon atoms, R₂ is a hydrogen or methyl group, n is a number from 1 to 3, R₃ is selected from the group consisting of hydrogen and an alkyl group having 1 to 5 carbon atoms, R₄ is selected from the group consisting of hydrogen and an alkyl group having 1 to 4 carbon atoms, and R₅ is a hydrogen, methyl, or ethyl group, and R₆ is an alkyl group having 1 to 18 carbon atoms.

20. The method according to claim 10, wherein the benzoate ester is incorporated into a polymer material.
